(19) United States
(12) Patent Application Publication
(10) Pub. No.: US 2021/0205640 A1
(43) Pub. Date: Jul. 8, 2021

(54) PORTABLE GAS SUPPLY DEVICE AND RESPIRATORY ASSISTANCE SYSTEM

(71) Applicant: KUNSHAN YUANSHAN TIANDI SOFTWARE TECHNOLOGY CO., LTD., Suzhou, Jiangsu (CN)

(72) Inventor: Yixin SUN, Suzhou (CN)

(73) Assignee: KUNSHAN YUANSHAN TIANDI SOFTWARE TECHNOLOGY CO., LTD., Suzhou, Jiangsu (CN)

(21) Appl. No.: 17/056,617

(22) PCT Filed: May 17, 2019
(86) PCT No.: PCT/CN2019/087492

§ 371 Date: Nov. 18, 2020

(30) Foreign Application Priority Data
May 21, 2018 (CN) 201810493015.4

(51) Int. Cl. A62B 7/02; A62B 9/02; A62B 18/10; A62B 18/02

(52) U.S. Cl. CPC A62B 7/02; A62B 18/025; A62B 18/10; A62B 9/02

(57) ABSTRACT

The present application provides a portable gas supply device and a respiratory assistance system. The portable gas supply device comprises a gas source and a gas transport unit; the gas source is configured to provide purified air; the gas transport unit comprises a gas transport mechanism, a buffer airbag, and a first gas check mechanism; the gas transport mechanism has a gas passage, an inlet end of the gas passage being connected to the gas source, and an outlet end thereof being used for outputting the purified air; the buffer airbag is communicated with the gas passage or the gas source; the first gas check mechanism is mounted in the gas passage and is configured to be in one-way conduction from the inlet end to the outlet end.

### CROSS-REFERENCE TO RELATED APPLICATIONS

The present disclosure claims priority to Chinese Patent Application No. 2018104930154, filed with the Chinese Patent Office on May 21, 2018, entitled “Portable Gas Supply Device and Respiratory Assistance System”, the contents of which are incorporated herein by reference in entirety.

### TECHNICAL FIELD

The present disclosure relates to a portable gas supply device and a respiratory assistance system.

### BACKGROUND ART

The current portable air purifying apparatuses mainly include masks, especially masks having a filtering function. Such mask itself has a filtering property. When air is being inhaled through the mouth and nose, negative pressure formed at the mouth and nose causes air to pass through and be filtered by the mask. Although the air is purified, breathing resistance is increased. The resistance caused by the mask will increase burden on the breather. Therefore, it is more difficult to run or exercise or perform other physical exertion activities while wearing a mask on a hazy or smoggy day. A human body performing physical exertion activities must become short of breath and can no longer bear the additional resistance caused by wearing of a mask. Therefore, people who want to run or exercise or perform other physical exertion activities in severe hazy outdoor environments have to give up such physical exertion activities or suffer from haze or smog during the activities.

Even in the case of running or doing exercise indoors, many gyms do not provide air purifiers to purify the air. If the local area has been invaded by haze, haze still exists in the indoor space although the indoor space is small and it seems that no haze is visible therein. Therefore, people who are running or doing exercise indoors are actually still suffering.

There are also people who work in places such as dusty mining areas and factories with severe air pollution. These places have severe air pollution. Although workers wear masks, they are actually working with much effort, and thus wearing masks will also increase burden on the users in inhalation. Many workers will not wear masks in order to work against time, or their masks have been used for a long time and are not replaced in time. Thus, the workers are deeply poisoned by polluted air.

The edge of a traditional mask is unlikely to perfectly fit a person's face, and an air leak(s) will be created where the edge of the mask does not fit the face. When the human body inhales, unpurified air will enter the respiratory system of the human body from the air leak(s) because the outside air is inhaled from the air leak(s) at the edge with less resistance than that in the case where the outside air is filtered and inhaled through the mask. As a result, the air purifying effect of the mask will be reduced. Users may feel at ease after wearing masks, but in fact they may have inhaled a lot of unpurified air unnoticeably.

### SUMMARY

An object of the present disclosure includes providing a portable gas supply device and a respiratory assistance system for providing purified air to a user.

The present disclosure includes the following technical solutions.

A portable gas supply device includes a gas source and a gas delivery unit, wherein the gas source is configured to provide purified air; and the gas delivery unit includes a gas delivery mechanism, a temporary storage balloon, and a first gas check mechanism;

the gas delivery mechanism has a gas passage, an inlet end of the gas passage is connected to the gas source, and an outlet end of the gas passage is configured to output purified air;

the temporary storage balloon communicates with the gas passage or the gas source, the first gas check mechanism is mounted at the gas passage, and the first gas check mechanism is configured for one-way connection from the inlet end to the outlet end.

Optionally, in an embodiment of the present disclosure, the gas delivery mechanism includes a first duct, the first gas check mechanism is mounted in the first duct, the temporary storage balloon is mounted at a side wall of the first duct or at a gas outlet end of the gas source, and the temporary storage balloon has an air inlet and an air outlet which are independent of each other or formed by a same one port.

Optionally, the gas delivery mechanism includes a first duct, the first gas check mechanism is mounted in the first duct, and the temporary storage balloon is mounted at a position closer to the gas source than the first gas check mechanism.

Optionally, in an embodiment of the present disclosure, the first gas check mechanism is mounted at a gas inlet end, a gas outlet end or a middle portion of the first duct.

Optionally, in an embodiment of the present disclosure, the gas delivery mechanism includes a first duct and a second duct, the temporary storage balloon has an air inlet and an air outlet, the air outlet of the temporary storage balloon is connected to the first duct, the first gas check mechanism is mounted at the first duct, and the second duct has one end connected to the air inlet of the temporary storage balloon and the other end connected to the gas source.

Optionally, in an embodiment of the present disclosure, the air inlet and the air outlet may be arranged at opposite sides of the temporary storage balloon, or adjacent to each other or at the same side of the temporary storage balloon.

Optionally, in an embodiment of the present disclosure, the temporary storage balloon is arranged in a hidden manner at or beside the gas source.

Optionally, in an embodiment of the present disclosure, a protective shell may be arranged, and the temporary storage balloon is arranged in a hidden manner in the protective shell, for example.

Optionally, in an embodiment of the present disclosure, the gas source includes an air storage tank configured for storing purified air or an air purifying device.

Optionally, in an embodiment of the present disclosure, the temporary storage balloon is made of a flexible and airtight material.

Optionally, in an embodiment of the present disclosure, the first gas check mechanism includes a check structure.

The present disclosure further provides a respiratory assistance system, which includes an isolation mask configured for covering the mouth and nose, a second gas check mechanism, and any of the portable gas supply devices described above, wherein the isolation mask is made of a material capable of isolating a gas, the isolation mask has an inhalation port and an exhalation port, the inhalation port is connected to the outlet end of the gas passage, and the second gas check mechanism is configured to allow the gas to exit the isolation mask in a one-way direction from the exhalation port.

Optionally, in an embodiment of the present disclosure, a sealing strip is arranged on an edge of the isolation mask, i.e., a portion of the isolation mask that is in contact with the skin of the face, and the sealing strip is made of an elastic and softer material such as silicone or rubber.

Optionally, in an embodiment of the present disclosure, the second gas check mechanism includes a check structure, and the check structure is mounted to the isolation mask and located at the exhalation port.

Optionally, in an embodiment of the present disclosure, the second gas check mechanism includes a check structure and a gas exhaust tube, the gas exhaust tube is mounted at the exhalation port of the isolation mask, and the check structure is mounted in the gas exhaust tube or at the exhalation port of the isolation mask.

Optionally, in an embodiment of the present disclosure, the isolation mask is made of an airtight hard or soft material.

The present disclosure includes, for example, the following advantageous effects: the technical problems to be solved by the portable gas supply device and the respiratory assistance system obtained by the above design in the present disclosure are not mentioned or disclosed in the prior art. The problem of short supply or low utilization of purified air is proposed for the first time by the inventor of the present disclosure in consideration of different characteristics of the rhythmic respiration of a human body and the generation of a gas by the gas source at a constant rate. In the portable gas supply device of this embodiment, the temporary storage balloon and the first gas check mechanism are used in close cooperation to reduce waste of clean air caused by a mismatch between supply and demand rhythms, thereby saving cost and prolonging its use time. Moreover, this gas supply device allows a user to breathe with less difficulty to avoid hypoxia in the user when wearing the mask for a long time, as compared with traditional masks.

### BRIEF DESCRIPTION OF DRAWINGS

In order to more clearly illustrate technical solutions of embodiments of the present disclosure, drawings required for use in the embodiments will be described briefly below. It is to be understood that the drawings below are merely illustrative of some embodiments of the present disclosure, and therefore should not be considered as limiting its scope. It will be understood by those of ordinary skill in the art that other relevant drawings can also be obtained from these drawings without any inventive effort.

FIG. 1 is a schematic view of a portable gas supply device according to embodiments of the present disclosure;

FIG. 2 is a schematic view of a temporary storage balloon and a first gas check mechanism which is a check mechanism in a swing structure/form, in an exhalation phase;

FIG. 3 is a schematic view of a temporary storage balloon and a first gas check mechanism which is a check mechanism in a swing structure/form, in an inhalation phase;

FIG. 4 is a schematic view of a portable gas supply device with a first gas check mechanism in a flap structure (valve structure) according to embodiments of the present disclosure, which is in the exhalation phase;

FIG. 5 is a schematic view of a portable gas supply device with a first gas check mechanism in a flap structure according embodiments of the present disclosure, which is in the inhalation phase;

FIG. 6 is a schematic view of a portable gas supply device with a first gas check mechanism in a flap structure according to embodiments of the present disclosure, which is in the exhalation phase;

FIG. 7 is a schematic view of a portable gas supply device with a first gas check mechanism in a flap structure/form according to embodiments of the present disclosure, which is in the inhalation phase;

FIG. 8 is a schematic view of a first gas check mechanism in a piston structure/form, in the exhalation phase;

FIG. 9 is a schematic view of a first gas check mechanism in a piston structure/form, in the inhalation phase; and

FIG. 10 is a schematic view of a respiratory assistance system according to embodiments of the present disclosure.

### REFERENCE NUMERALS

100—portable gas supply device; 120—temporary storage balloon; 130—first gas check mechanism; 140—first duct; 150—second duct; 200—respiratory assistance system; 210—isolation mask; 220—second gas check mechanism.

### DETAILED DESCRIPTION OF EMBODIMENTS

In order to further clarify the objects, technical solutions, and advantages of the embodiments of the present disclosure, the technical solutions of the embodiments of the present disclosure will be described below clearly and completely with reference to the drawings of the embodiments of the present disclosure. It is apparent that the embodiments to be described are some, but not all of the embodiments of the present disclosure. All the other embodiments obtained by those of ordinary skill in the art in light of the embodiments of the present disclosure without inventive efforts will fall within the scope of the present disclosure as claimed. Thus, the following detailed description of the embodiments of the present disclosure, as represented in the figures, is not intended to limit the scope of the present disclosure as claimed, but is merely representative of selected embodiments of the present disclosure. All the other embodiments obtained by those of ordinary skill in the art in light of the embodiments of the present disclosure without inventive efforts will fall within the scope of the present disclosure as claimed.

In the description of the present disclosure, it should be understood that the terms such as “up”, “down”, “inside”, and “outside” indicate the orientation or positional relationships shown based on the figures, and these terms are intended only to facilitate the description of the present disclosure and simplify the description, but not intended to indicate or imply that the referred devices or elements must be in a particular orientation or constructed or operated in the particular orientation, and therefore should not be construed as limiting the present disclosure.

In addition, the terms “first” and “second” are used for descriptive purposes only, and should not be understood as an indication or implication of relative importance or an implicit indication of the number of the indicated technical features. Therefore, a feature defined with the terms “first” and “second” may explicitly or implicitly include one or more such features. In the description of the present disclosure, “a plurality” means two or more unless otherwise expressly and specifically defined.

In the present disclosure, the terms “mounted”, “coupled”, “connected”, “fixed”, and the like should be understood broadly unless otherwise expressly specified or defined. For example, connection may be fixed connection or detachable connection or integral connection, or may be direct coupling or indirect coupling via an intermediate medium or internal communication between two elements or mutual interaction between two elements. The specific meanings of the above-mentioned terms in the present disclosure can be understood by those of ordinary skill in the art according to specific situations.

In the present disclosure, unless otherwise expressly specified or defined, a first feature “on (or above)” or “below” a second feature may include a case where the first and second features are in direct contact, and may also include a case where the first and second features are not in direct contact, but are in contact via an additional feature therebetween. Moreover, that a first feature is “on”, “above”, or “over” a second feature is meant to include a case where the first feature is directly above or obliquely above the second feature, or merely means that the first feature is at a level height higher than the second feature. That a first feature being “below”, “under”, or “underneath” a second feature is meant to include a case where the first feature is directly below or obliquely below the second feature, or merely means that the first feature is at a level height lower than the second feature.

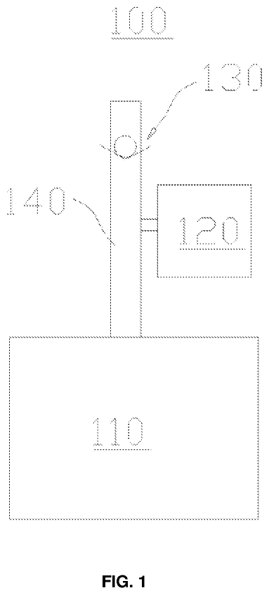

As shown in FIG. 1, this embodiment provides a portable gas supply device 100, which includes a gas source 110 and a gas delivery unit. The gas delivery unit includes a gas delivery mechanism 140, a temporary storage balloon 120, and a first gas check (or non-return) mechanism 130. This portable gas supply device 100 may be used by personnel in various application scenarios, including but not limited to medical personnel, cooks, various decoration workers, and so on. For example, it may be carried in an environment with poor air quality for, including but not limited to, providing purified air to a user at work or during exercise. If the user is in an environment that is not suitable for air purification over a large area, this portable gas supply device 100 is a better choice. Optionally, this portable gas supply device 100 may also be used by a user during running or walking.

The gas source 110 described in the present disclosure is configured to provide purified air. The gas source 110 may be a compressed air tank storing purified air, or a portable air purifier, or even a combination thereof.

The time during which the portable gas supply device 100 is usable may be limited by the carrying capacity of the air tank, or limited by the duration of a power source or the service life of an air filter element (air filter core). Therefore, the applicant of the present disclosure hopes that the portable air purifier can achieve high utilization rate of purified air.

In the portable gas supply device 100 according to this embodiment, the gas delivery mechanism has a gas passage, the inlet end of the gas passage is connected to the gas source 110, and outlet end of the gas passage is used for outputting purified air. The temporary storage balloon 120 communicates with the gas passage or the gas source. The first gas check mechanism 130 is mounted at the gas passage. The first gas check mechanism 130 is configured for one-way connection from the inlet end to the outlet end.

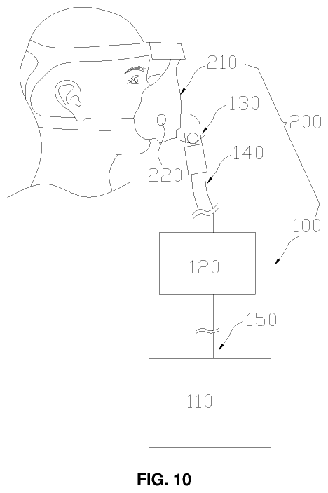

As shown in FIG. 10, when the outlet end of the gas passage is connected to a face mask or an oronasal mask or an isolation mask or the like, a respiratory assistance system 200 capable of providing purified air can be formed.

The first gas check mechanism 130 has the function of preventing backflow of a gas, especially preventing the gas exhaled by the user from flowing back to the gas supply device so as to avoid contamination of the gas supply device by the gas exhaled by the user. When the first gas check mechanism 130 is in a closed state, outflow of the gas from the first duct 140 can be blocked. The power for opening the first gas check mechanism 130 may come from a pressure difference formed between pressures at the two sides of the first gas check mechanism 130 when the user inhales.

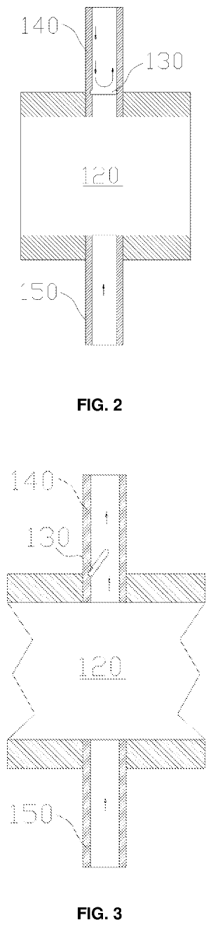

As shown in FIG. 3, when the user is inhaling, the pressure produced by the user's inhalation causes the first gas check mechanism 130 to be in an open state, so that purified air flows from the gas source 110 or the temporary storage balloon 120 to the nose and mouth of the user. Because the inhalation speed is higher than the speed at which the gas source produces clean air/purified air, the purified air temporarily stored in the balloon decreases and the balloon shrinks. As shown in FIG. 2, when the user is exhaling, the pressure produced by the user's exhalation causes the first gas check mechanism 130 to be in the closed state, and the user will not inhale purified air from the gas source 110 at this time. However, the purified air is still continuously released from the gas source 110. Eventually, the purified air not inhaled by the user will be temporarily stored in the balloon 120, and the balloon is expanded. The temporary storage balloon 120 is used for storing this part of the purified air. If the first gas check mechanism 130 and the temporary storage balloon 120 are not arranged, the purified air generated during the exhalation phase will be wasted.

Optionally, in an embodiment of the present disclosure, the gas delivery mechanism may include a first duct 140. Here, the first gas check mechanism 130 is mounted in the first duct 140. The temporary storage balloon 120 is mounted at a side wall of the first duct 140 or at the gas source 110. The temporary storage balloon 120 has an air inlet and an air outlet which are independent of each other or formed by a same one port. In other words, the side wall of the first duct 140 may be provided with a first hole and a second hole distributed in the axial direction, and the temporary storage balloon 120 is provided with an air inlet and an air outlet individually corresponding to the first hole and the second hole. The first hole and the second hole are hermetically connected to the corresponding inlet and outlet and make the temporary storage balloon 120 communicate with the internal space of the first duct 140. Alternatively, the side wall of the first duct 140 may be provided with only one of the first hole and the second hole, and the temporary storage balloon 120 is provided with only one opening corresponding to the hole in the side wall of the first duct 140.

The first gas check mechanism 130 may be mounted at any position of the first duct 140, for example, at the gas inlet end, the gas outlet end or the middle portion of the first duct 140, or even mounted between the gas outlet end of the first duct and the isolation mask, in order to prevent the exhaust gas exhaled by the user from flowing back to contaminate the first duct.

Optionally, the first gas check mechanism 130 is mounted in the first duct 140, and the temporary storage balloon 120 is mounted at a position closer to the gas source than the first gas check mechanism 130.

Optionally, the first gas check mechanism 130 is located in the first duct 140 and between the gas outlet end and the first hole (provided that the first hole is closer to the gas outlet end than the second hole) or at the gas outlet end of the first duct. In this way, when the first gas check mechanism 130 is closed, purified air can enter the temporary storage balloon 120, and the exhaust gas exhaled by the user will be blocked by the first gas check mechanism 130 and will neither flow back to the temporary storage balloon 120 nor enter the gas source 110 to be mixed with purified air.

Optionally, in an embodiment of the present disclosure, the gas delivery mechanism includes at least a first duct 140 and a second duct 150. The temporary storage balloon 120 has an air inlet and an air outlet. The air outlet of the temporary storage balloon 120 is connected to the first duct 140. The first gas check mechanism 130 is mounted at any position of the first duct. The second duct 150 has one end connected to the air inlet of the temporary storage balloon 120 and the other end connected to the gas source 110.

The air inlet and the air outlet may be arranged at the opposite sides of the balloon, or adjacent to each other or at the same side of the balloon, and are optionally arranged on the opposite sides. The first gas check mechanism 130 may be mounted at any position of the first duct 140. When the first gas check mechanism 130 is in the closed state, entry of the gas exhaled by the user into the temporary storage balloon 120 can be blocked. The first gas check mechanism 130 may be mounted at either of the two end or at the middle of the first duct 140. The first gas check mechanism 130 may be mounted at the place where the first duct 140 is connected with the temporary storage balloon 120. In this case, it is equivalent to being mounted at the end of the first duct 140.

The temporary storage balloon 120 may be positioned close to the gas outlet end of the gas supply device, or close to the gas source 110, or may be hidden at the gas source 110. A protective shell may be arranged such that the gas source and the temporary storage balloon 120 are hidden in the protective shell. When used by the user, the gas source 110 may optionally be secured to the user's body in a detachable connection manner, for example, strapped around the shoulder or waist of the user, so that the position of the gas source 110 is relatively stable. When the temporary storage balloon 120 is arranged in a hidden manner in the protective shell, it can be ensured that the temporary storage balloon 120 is less or not affected by the outside, including affecting the normal shrinkage or expansion of the balloon or avoiding damage of the temporary storage balloon 120 is, or the like. When the temporary storage balloon 120 is hidden at the gas source 110, in other words, when the temporary storage balloon 120 is arranged in the shell at the gas source 110, the gas delivery mechanism may include the first duct 140, or may be formed to include the first duct 140 and the second duct 150.

Optionally, the gas source 110 includes an air tank storing purified air or an air purifying device. The air purifying device may include components such as a housing, a fan that forms an air flow, a filter membrane structure or a filter net that purifies air, and a power source. The air purifying device may be a miniaturized air purifying device so as to be easily carried by a person. Here, the filter net may include a HEPA filter net, which has a better air purification effect.

Optionally, the temporary storage balloon 120 is made of a flexible and airtight material. For example, the temporary storage balloon 120 may be made of a resin film, or rubber, or a fabric coated with an airtight coating, or the like. On the one hand, the main body of the temporary storage balloon 120 may be made of a film and combined with some additional rigid connection structures to facilitate the connection of the balloon to the duct. On the other hand, the temporary storage balloon 120 may include only a film. For example, the temporary storage balloon 120 is connected to the duct by bonding or other means, with the connected portion being sealed. When being made of a flexible and airtight material, especially a film, the temporary storage balloon 120 is expandable without overcoming greater resistance. In other words, it is easier to inflate the temporary storage balloon. If the temporary storage balloon 120 is inflated against a resistance which is too large or even greater than the restoring force of the check structure, the check structure may be pushed to be open. Of course, the temporary storage balloon 120 may also be made of a rubber film, but the rubber film should not be designed with too large elasticity.

Optionally, the first gas check mechanism 130 may include a check structure. The first gas check mechanism 130 may have a flap structure to achieve one-way connection. The first gas check mechanism 130 may automatically return to the original position or be automatically closed under gravity or an elastic force. When the user inhales, a gas flow and pressure difference are caused, so that the first gas check mechanism 130 is automatically opened.

The technical problems to be solved by this portable gas supply device 100 are not mentioned or disclosed in the prior art. The problem of short supply or low utilization of purified air is proposed for the first time by the applicant of the present disclosure in consideration of different characteristics of the rhythmic respiration of a human body and the generation of a gas by the gas source at a constant rate. In the portable gas supply device of this embodiment, the temporary storage balloon and the first gas check mechanism are used in close cooperation to reduce waste of clean air caused by a mismatch between supply and demand rhythms, thereby saving cost and prolonging its use time. Moreover, this gas supply device allows a user to breathe with less difficulty to avoid hypoxia in the user when wearing the mask for a long time, as compared with traditional masks.

As shown in FIG. 10, this embodiment also provides a respiratory assistance system 200, which includes an isolation mask 210 for covering the mouth and nose, a second gas check mechanism 220, and any one of the portable gas supply devices 100 described above. The isolation mask 210 is made of a material capable of isolating a gas. The isolation mask 210 has an inhalation port and an exhalation port. The inhalation port is connected to the outlet end of the gas passage 140. The second gas check mechanism 220 is configured to allow the gas to exit the isolation mask 210 in a one-way direction from the exhalation port. The second gas check mechanism 220 is closed when the user inhales, and opened when the user exhales.

The isolation mask 210 has the main function of isolating, rather than purifying or filtering, air, for example, isolating the space around the mouth and nose from the outside, so as to facilitate the inhalation of the purified air provided by the gas source 110 into the body and avoid inhalation of unpurified air from the outside. Although the isolation mask 210 can isolate a gas, the gas is not necessarily isolated completely and absolutely. The incomplete and non-absolute isolation is caused mainly by two factors. On the one hand, it is difficult to completely seal the face with the isolation mask 210. The edge of the mask cannot fit the face perfectly, and an air leak(s) will be created where the edge of the mask does not fit the face. The purifying effect of the mask will be affected by entry of unpurified air from the air leak(s). After all, outside air is inhaled from the air leak(s) at the edge with less obstruction than that in the case where the outside air is filtered and inhaled through the mask. A user may feel at ease after wearing a mask, but in fact he or she may have inhaled a lot of waste gas unnoticeably. If the gas source actively supplies air, the entire respiratory assistance system can be maintained at an air pressure higher than the outside by adjusting and increasing the air supply speed, thereby keeping the air pressure in the isolation mask higher than the outside. In this way, the gas in the isolation mask is always in a state of flowing outward, thereby preventing inhalation of outside air by the user from the air leak(s) at the edge.

In order to improve airtightness, a sealing strip is optionally arranged at the edge of the isolation mask 210, i.e., its portion that is in contact with the skin of the face. The sealing strip may be made of an elastic and softer material such as silicone or rubber. On the other hand, an isolation mask 210 with a better air isolation effect may be manufactured by using medical plastic or other hard materials. The isolation mask 210 itself may not completely isolate air. The isolation mask 210 may be made of some materials with poor air permeability, such as some densely woven fabrics or the like.

Optionally, the isolation mask 210 is made of an airtight hard or soft material. Optionally, the isolation mask 210 is made of a hard material or a combination of hard and soft materials, so that a certain gap can be maintained between the isolation mask 210 and the nose and mouth. When a gap is maintained between the isolation mask 210 and the nose and mouth, purified air is more easily obtained from the gas source 110. A soft material may have its hardness improved by increasing its thickness, or may be used in combination with a hard material and supported by means of a skeleton structure of the hard material.

The inhalation port of the isolation mask 210 is connected to the outlet end of the gas passage 140, so that purified air can be obtained from the gas source 110.

The second gas check mechanism 220 is opened when the user breathes out, and closed when the user breathes in. The second gas check mechanism 220 is open or connected in a determined direction. In other words, the gas can only be exhaled from the isolation mask to the outside and cannot be inhaled into the isolation mask from the outside.

Optionally, the second gas check mechanism 220 includes a check structure. The check structure is mounted to the isolation mask 210 and located at the exhalation port. It should be noted that there may be one or more inhalation ports, and similarly, there may be one or more exhalation ports.

Optionally, the second gas check mechanism 220 includes a check structure and a gas exhaust tube. The gas exhaust tube is mounted at the exhalation port of the isolation mask. The check structure is mounted in the gas exhaust tube or between the gas exhaust tube and the exhalation port of the isolation mask.

There are many choices for the specific structure of the second gas check mechanism 220, which may be designed with reference to the structure of the first gas check mechanism 130 and may be the same as or different from the structure of the first gas check mechanism 130.

The first gas check mechanism 130 and the second gas check mechanism 220 may arbitrarily be selected from any one of check structures described below.

Optionally, as shown in FIGS. 2 and 3, the check structure may have a swing structure, and the check structure has a valve plate movably connected to the inner wall of the first duct 140. The valve plate may be maintained in a state shown in FIG. 2 by means of gravity or an elastic member such as a spring or a torsion spring or the like, so that the valve plate is in a normally closed state. When the first gas check mechanism 130 employs a swing structure, the valve plate is opened during inhalation and the valve plate is closed during exhalation. When the second gas check mechanism 220 employs a swing structure, the valve plate is closed during inhalation and the valve plate is opened during exhalation.

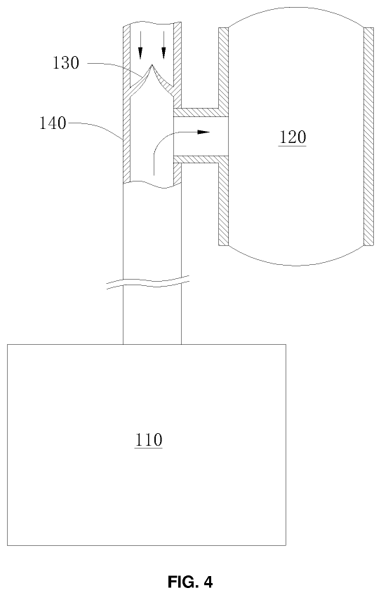

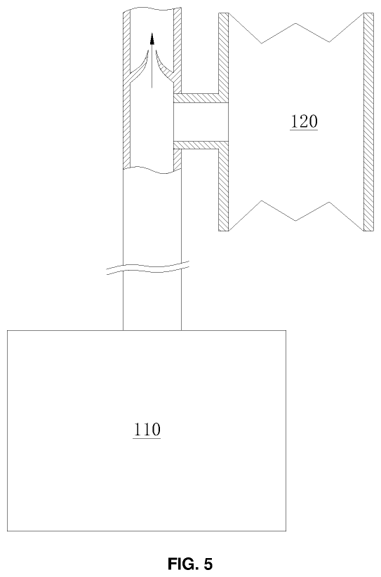

As shown in FIGS. 4 and 5, the check structure may have a flap structure with elastic flaps that are automatically movable close to each other to block the first duct 140. When the first gas check mechanism 130 employs a flap structure, the flaps are opened during inhalation and the flaps are closed during exhalation. When the second gas check mechanism 220 employs a flap structure, the flaps are closed during inhalation and the flaps are opened during exhalation.

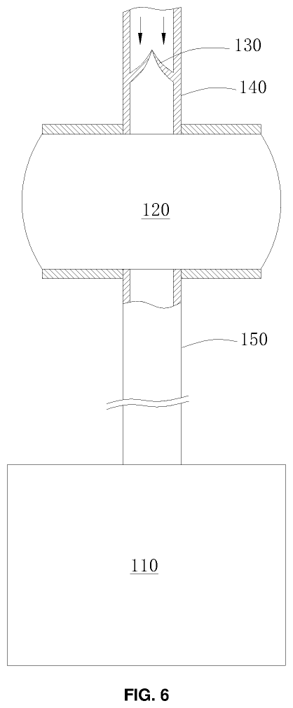

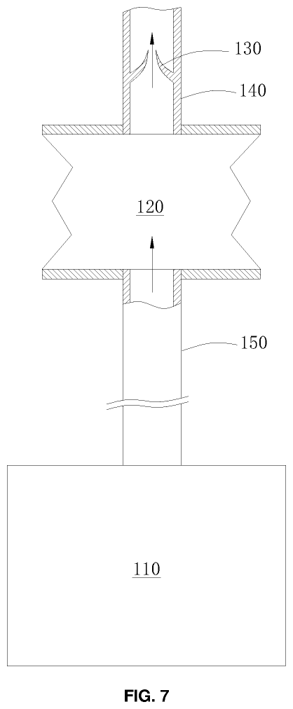

In the portable gas supply device shown in FIGS. 4 and 5, the temporary storage balloon 120 is connected to the side wall of the first duct 140. As an optional embodiment, a portable gas supply device shown in FIGS. 6 and 7 includes a first duct 140 and a second duct 150, a temporary storage balloon 120 is connected between the first duct 140 and the second duct 150, and a first gas check mechanism 130 is mounted in the first duct 140.

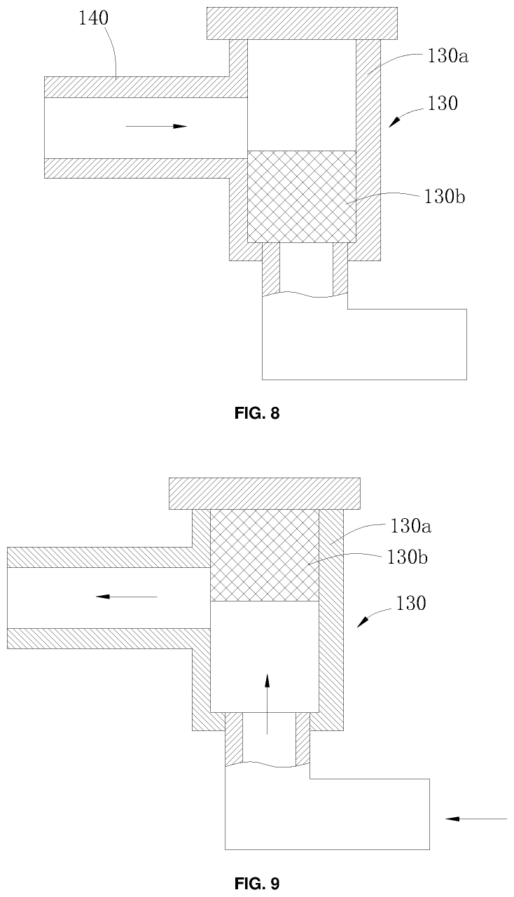

An embodiment of the present disclosure also provides an optional check structure. Referring to FIGS. 8 and 9, the check structure has a piston structure which includes a valve body 130a and a valve core 130b. The valve core 130b is movably mounted in a valve cavity (not marked) of the valve body 130a. When the first gas check mechanism 130 employs a piston structure, the valve body 130a is connected to the first duct 140, and the valve cavity of the valve body 130a communicates with the duct cavity of the first duct 140. The valve core 130b usually stays at the air inlet of the valve cavity (i.e., an opening near the bottom of the valve cavity in FIG. 8) under the action of gravity. When the first gas check mechanism 130 employs a piston structure, the valve core 130b moves up and the first gas check mechanism 130 is opened during inhalation, and the valve core 130b moves down and the first gas check mechanism 130 is closed during exhalation. When the second gas check mechanism 220 employs a piston structure, the valve core 130b moves up and the second gas check mechanism 220 is opened during exhalation, and the valve core 130b moves down and the second gas check mechanism 220 is closed during inhalation. Here, the valve core of the piston structure may be a ball plug or a plunger.

In an embodiment of the present disclosure, a plurality of ducts, balloons, and check mechanisms may be provided.

In the portable gas supply device 100 according to this embodiment, purified air is forced into the isolation mask 210 (oronasal mask), so that the breather can inhale without burden. Purified air can be continuously supplied from the gas source 110 of the portable gas supply device 100. When the user is in the exhalation phase, the purified air supplied from the gas source 110 is stored in the temporary storage balloon 120 to avoid waste. When the user is in the inhalation phase, the purified air stored in the balloon can be used. In this way, the problem of a mismatch between the constant supply of air from the gas source and rhythm between the alternate inhalation and exhalation of air by the user is solved.

The above description is merely illustrative of preferred embodiments of the present disclosure and is not intended to limit the present disclosure. It will be understood by those skilled in the art that various modifications and variations may be made to the present disclosure. Any modifications, equivalent alternatives, improvements and so on made within the spirit and principle of the present disclosure are intended to be encompassed within the scope of protection of the present disclosure.

### INDUSTRIAL APPLICABILITY

In the portable gas supply device of the present disclosure, the temporary storage balloon and the first gas check mechanism are used in close cooperation to reduce waste of clean air caused by a mismatch between supply and demand rhythms, thereby saving cost and prolonging its use time. Moreover, this gas supply device allows a user to breathe with less difficulty to avoid hypoxia in the user when wearing the mask for a long time, as compared with traditional masks.

1. A portable gas supply device, comprising a gas source and a gas delivery unit, wherein the gas source is configured to provide purified air, and the gas delivery unit comprises a gas delivery mechanism, a temporary storage balloon, and a first gas check mechanism;
the gas delivery mechanism has a gas passage, an inlet end of the gas passage is connected to the gas source, and an outlet end of the gas passage is configured to output purified air; and
the temporary storage balloon communicates with the gas passage or the gas source, the first gas check mechanism is mounted at the gas passage, and the first gas check mechanism is configured for one-way connection from the inlet end to the outlet end.

2. The portable gas supply device according to claim 1, wherein the gas delivery mechanism comprises a first duct, the first gas check mechanism is mounted in the first duct, the temporary storage balloon is mounted at a side wall of the first duct or at the gas source, and the temporary storage balloon has an air inlet and an air outlet which are independent of each other or formed by a same one port.

3. The portable gas supply device according to claim 1, wherein the gas delivery mechanism comprises a first duct, the first gas check mechanism is mounted in the first duct, and the temporary storage balloon is mounted at a position closer to the gas source than the first gas check mechanism.

4. The portable gas supply device according to claim 2, wherein the first gas check mechanism is mounted at a gas inlet end, a gas outlet end or a middle portion of the first duct.

5. The portable gas supply device according to claim 1, wherein the gas delivery mechanism comprises a first duct and a second duct, the temporary storage balloon has an air inlet and an air outlet, the air outlet of the temporary storage balloon is connected to the first duct, the first gas check mechanism is mounted at the first duct, and the second duct has one end connected to the air inlet of the temporary storage balloon and the other end connected to the gas source.

6. The portable gas supply device according to claim 5, wherein the air inlet and the air outlet are arranged at opposite sides of the temporary storage balloon, or adjacent to each other or at a same side of the temporary storage balloon.

7. The portable gas supply device according to claim 1, wherein the temporary storage balloon is arranged in a hidden manner at or beside the gas source.

8. The portable gas supply device according to claim 1, wherein the portable gas supply device comprises a protective shell in which the temporary storage balloon is arranged in a hidden manner.

9. The portable gas supply device according to claim 1, wherein the gas source comprises an air tank configured for storing the purified air, or an air purifying device.

10. The portable gas supply device according to claim 1, wherein the temporary storage balloon is made of a flexible and airtight material.

11. The portable gas supply device according to claim 1, wherein the first gas check mechanism comprises a check structure, and the check structure is any one of a check structure in a swing form, a check structure in a piston form and a check structure in a flap form.

12. A respiratory assistance system, comprising an isolation mask configured for covering a mouth and a nose, a second gas check mechanism, and the portable gas supply device according to claim 1, wherein the isolation mask is made of a material capable of isolating a gas, the isolation mask has an inhalation port and an exhalation port, the inhalation port is connected to the outlet end of the gas passage, and the second gas check mechanism is configured to allow the gas to exit the isolation mask in a one-way direction from the exhalation port.

13. The respiratory assistance system according to claim 12, wherein a sealing strip is arranged at an edge of the isolation mask, i.e., a portion of the isolation mask that is in contact with a skin of a face, and the sealing strip is made of an elastic and softer material, such as silicone or rubber.

14. The respiratory assistance system according to claim 12,
wherein the second gas check mechanism comprises a check structure, and the check structure is mounted to the isolation mask and located at the exhalation port; or
the second gas check mechanism comprises a check structure and a gas exhaust tube, the gas exhaust tube is mounted at the exhalation port of the isolation mask, and the check structure is mounted in the gas exhaust tube or at the exhalation port of the isolation mask.

15. The respiratory assistance system according to claim 12, wherein the check structure is any one of a check structure in a swing form, a check structure in a piston form, and a check structure in a flap form.

16. The respiratory assistance system according to claim 12, wherein the isolation mask is made of an airtight hard or soft material, or made of a combination of a hard material and a soft material.

17. The portable gas supply device according to claim 3, wherein the first gas check mechanism is mounted at a gas inlet end, a gas outlet end or a middle portion of the first duct.

18. The portable gas supply device according to claim 2, wherein the temporary storage balloon is arranged in a hidden manner at or beside the gas source.

19. The portable gas supply device according to claim 2, wherein the portable gas supply device comprises a protective shell in which the temporary storage balloon is arranged in a hidden manner.

20. The portable gas supply device according to claim 2, wherein the first gas check mechanism comprises a check structure, and the check structure is any one of a check structure in a swing form, a check structure in a piston form and a check structure in a flap form.
